(19) United States
(12) Patent Application Publication
(10) Pub. No.: US 2014/0065616 A1
(43) Pub. Date: Mar. 6, 2014

(54) Isoltation of Factors Associated with Nucleic Acid

(71) Applicant: Hanpeng Xu, Los Angeles, CA (US)

(72) Inventor: Hanpeng Xu, Los Angeles, CA (US)

(21) Appl. No.: 14/020,003

(22) Filed: Sep. 6, 2013

(30) Foreign Application Priority Data
Sep. 6, 2012 (CH) 201210326387.0

(51) Int. Cl. C12Q 1/68; G01N 33/68

(52) U.S. Cl. CPC C12Q 1/6876; G01N 33/68
USPC 435/6.11; 435/6.12; 435/7.1

(57) ABSTRACT

Methods for screening and isolating peptide, polypeptide, protein complexes and non-coding nucleic acids that are associated with selected target genomic locus are provided. The methods comprise the steps of obtaining a sample that comprises a modified target genomic DNA sequence and one or more peptide, polypeptide, protein complexes and non-coding nucleic acids as with that DNA sequence. The target genomic locus DNA sequence which contain all the elements that enable it keeping its function independently in spite of their genomic position are modified by introducing one or more labeling and cutting sequences. These modified target genomic locus DNA sequences are amplified and purified. The purified modified target genomic locus DNA sequences are introduced into cells or animals and their functions are regulated as the same as original endogenous target genomic locus. The modified target sequence and the factors associated with it are crosslinked and selectively isolated.

### FIELD OF THE INVENTION

The invention relates to a method for isolation of factors associated with nucleic acid.

### DISCUSSION OF RELATED ART

Genomes are the entirety of an organism's hereditary information. Genomes are encoded either in DNA, or for many types of viruses in RNA. The genome includes both the genes and the non-coding sequences of the DNA/RNA.

A chromatin is the combination of DNA and protein complexes that make on the contents of the nucleus of a cell. Chromatin is only found in eukaryotic cells. Myriad proteins and non-coding nucleic acids associated with the genome contribute to its normal functions, which include packaging DNA into a smaller volume to fit within the cell, strengthening the DNA to allow mitosis, preventing DNA damage, controlling gene expression and DNA replication.

A gene is a locatable region of genomic sequence, corresponding to a unit of inheritance, which is associated with regulatory regions, transcribed regions, and or other functional sequence regions. The DNA for a given gene in eukaryotes is organized into exons and introns. The exons are those expressed sequences that become the mRNA, and the introns are those intervening sequences that are removed in the process of making a mature mRNA.

It is believed that the genomic DNA packaging level plays an important role in gene expression regulation. Highly expressed genes tend to exist in a low packaging state (euchromatic state), whereas silenced genes exist in a high packaging state (heterochromatic state). The level of packaging, also called condensation of the genomic DNA can vary between a lower packaged state, such as before the replication of DNA (G1 Phase) to a more condensed state, such as during cell division (M phase). The relative state of condensation, maintenance of this state and the transition between heterochromatin and euchromatin is believed to be mediated largely by a plurality of specialist proteins, polypeptide complexes, and RNAs.

Both endogenous and exogenous factors can make post translational modifications of factors associated with the DNA, and influence the transmission of information from a cell or multicellular organism to its descendants without changing the information being encoded in the nucleotide sequence of genes. This mechanism is called epigenetic mechanism.

In eukaryotic organisms, all the genes in the chromatin cannot be expressed at the same time. Gene expression must be tightly controller according to the developmental requirements of the cells and organisms. Epigenetic controls over chromatin organization and stability are essential for the normal and healthy functioning of a cell. Aberrant epigenetic modifications and decreases in chromatin stability are often seen in senescent, apoptotic or diseased cells, particularly in cancer cells.

The average length of eukaryotic gene is 27 kb, 85%, of gene lengths are less than 100 kb. In human genomes, the encoding sequence just occupies 1%, with 99% being non-coding sequences. It is believed that those non-coding sequences are responsible for the regulation of gene expression, although the underlying mechanisms are largely unknown at this time. In the non-coding sequences, the introns occupy 24%, and the remainder of the sequences is non-coding regulatory sequences and large amounts of repeated sequences.

The expression regulation of each gene could be carried out separately. Among the multiple steps during gene expression regulation, transcription plays an important role. The change of gene expression level depends on the binding of various transcriptional factors or other factors to various regulatory sequences that associate with that gene. The positions of those regulatory sequences associated with one gene are unpredictable. The distribution of the transcription factors and other factors, their types and amount and their direct or indirect interactions, both between them with the regulatory sequences and between themselves, form a complex regulation network and can only be verified by experiments. For one locus in the chromatin, the unique combination of the factors associated with it will determine the expression level of the genes located in that locus and the distribution of gene expression products in different tissue, or cells types at certain developmental stage. In a human genome, the factors that can directly bind DNA and regulate gene expression usually have DNA binding domains, the expected transcriptional factors number is more than 2600.

Due to their important role in chromatin function, it is of considerable importance to identify and characterize the multiple factors that are capable of exhibiting epigenetic activities, as well as those that are capable of interacting with chromatin and chromatin associated proteins. It would also be of great value to identify and characterize novel chromatin associated factors, not least to facilitate a better understanding of chromatin biology as a whole. In genomic research, it is a great challenge when studying factors that associate with a specific chromatin locus due to lack of an efficient, sensitive and specific method.

Currently, there are two methods used by researchers when studying chromatin locus associated factors, CHIP and PICH. The CHIP technique needs at least one antibody that recognizes one of the associated factors. After crosslinking the associated factors with their binding DNA, chromatin are fragmented and the antibody will bind and pull-down those recognized factors along with others factors binding on the same chromatin fragment. CHIP is antigen based, and will capture all the loci which bind to the same factor. CHIP is not a site-specific method, and the antibody is not always available. In many cases, the user has no idea what kind of factors are associating with certain chromatin locus.

The PICH technique is sequence based. After crosslinking, the associated factors with their binding DNA and chromatin are fragmented, a special designed probe with a complementary sequence to the target chromatin locus DNA is used to perform hybridization with chromatin fragments. The hybridized chromatin fragments are isolated and purified, and the factors associated with that chromatin sequence are assayed. Theoretically, this method could be used in any chromatin locus to obtain all the factors associated with that locus. However, the complex chromatin structure makes the optimization hybridization condition complicated. Since most genes in one cell only have two copies under normal conditions, to obtain enough material for isolation and further assay, the PICH method needs more than 10⁹ cells for one experiment, which form a practical challenge for most gene studies.

What is needed is another method to allow researchers to study the chromatin locus specific associated factors.

### SUMMARY OF THE INVENTION

To overcome the above mentioned difficulties in genomic research, especially in gene regulation research, the present invention provides an efficient method to screen, isolate and assay the factors that associated with a selected gene or genomic locus. A user can use this method in any locus or sequence in the genome at both cellular and tissue level. This invention provides a method to screen, isolate and assay factors associated with a target sequence, and this sequence could be coding or noncoding nucleic acid chain, either genomic or artificial type. Methods for screening and isolating peptide, polypeptide, protein complexes and non-coding nucleic acids that are associated with chromatin modified target genomic locus are also provided.

The methods comprise the steps of obtaining a sample that comprises modified target genomic DNA sequence and one or more peptide, polypeptide, protein complexes and non-coding nucleic acids associated with that target DNA sequence. The target genomic locus DNA sequence which contain all the elements that enable it keeping its function independently in spite of their genomic position are modified by introducing one or more labeling and cutting sequences. These modified target genomic locus DNA sequences are amplified and purified. The purified modified target genomic locus DNA sequences are introduced into cells or animals and their functions are regulated as the same as the original endogenous target genomic locus. The factors associated with the endogenous target sequence bind the introduced modified target sequences in the same way as they bind the endogenous target sequence. The introduced modified target sequences play as bait sequences to catch those factors associated with the endogenous locus. Contacting the modified target genomic locus DNA with factors that interact with the cutting sites makes double strand DNA breaks at the cutting sites. Partial or entire parts of the modified target genomic locus DNA and the peptide, polypeptide, protein complexes and non-coding nucleic acids associated with it are released from the cellular chromatin, isolated from the sample through centrifuge, immobilization molecules that bind to at least one component of the modified target DNA and its associated factors. Binding sites of the associated factors on the modified target genomic locus DNA are determined by sequencing, and the nature of associated factors are assayed with standard molecular methods. The methods of the invention are suited to identification of peptide, polypeptide, protein complex and non-coding RNAs including micro RNAs and snoRNAs that are associated with chromatin remodeling and gene expression. The method is suited to all eukaryotic cells.

This method includes the following essential steps:
(1) Target sequence selection: The target sequence could be either artificial synthesis nucleic sequence or a sequence in genome. For a gene function study, the target sequence may include all the encoding sequence and potential regulatory sequences. The range of the genome sequence length is between 10 kb-1 MB, usually between 20-300 kb to fit the insert size of vector.
(2) Target sequence modification: One or more manipulator sequences are introduced into certain sites of target sequence. These manipulator sequences are used to screen the positive clone, detect gene expression and be used as recognition and cutting sites of endonuclease or other artificial DNA cutter.
(3) Modified target sequences amplification: The modified target sequences are amplified in appropriate cell lines and purified. Purified modified target sequences are introduced into host cells and animals to make transgenic cell lines or transgenic animals.
(4) Modified target sequences function detection in vivo: The status of modified target sequences in transgenic cells or animals are detected by appropriate assay such as phenotype and/or through evaluating introduced marker.
(5) Modified target sequence and its associated factor isolation: Modified target sequences are crosslinked with the factors associated with them in the host transgenic cell lines or animals tissue. The modified target sequence are contacted with reagents that bind and cut DNA at introduced cutting sites, make one or more fragments from the modified target sequence between the introduced cutting sites.
(6) Modified target sequence fragments isolation and assay: The fragments derived from modified target sequence and the factors associated with them are isolated, the crosslink are reversed and factors and factor binding sites on the target sequence are assayed.

In step (2) in the above mentioned step 2, the target sequence modification includes introducing a selectable marker, reporter marker, and/or endonuclease site or a artificial cutter sites into certain positions in the target sequence. The introduced selectable marker or reporter marker can be antibiotic selectable marker and/or fluorescence proteins, or enzyme, which can replace partial or entire encoding sequence of the target locus. These selectable or reporter markers are used to monitor the target sequence status in the following steps. The introduced endonuclease site and/or artificial cutter site are extremely rare that only those endonucleases and artificial cutter which have long recognition sites are able to specifically bind and cut at those sites and make double strand DNA breaks and produce fragments between those sites. The introduced extreme rare endonucleases include but not limited to meganuclease, recombinases or other endonuclease that could specifically recognize the introduced sites and cut it to produce double strand DNA breaks fragments. The introduced recognition sites length of extreme rare endonuclease or artificial cutter are more than 10 bp to make sure they are unique in the whole genome. The method that introduces those modifications in the target sequence is standard molecular techniques and/or artificial assembly method.

Step (3) above is a large scale amplification of the modified target sequence. The modified target large genomic fragment is amplified in appropriate host bacteria, or produced through Gibson Assembly method. The amplified modified target sequences are purified with standard molecular method and shear force need to be avoided. These purified modified target sequences are introduced into cells using standard transfection method to obtain transgenic cell lines, both transient expression or stable cell lines and/or introduced into animals using transgenic techniques. The copy numbers in the transgenic cell line or transgenic animals are verified using southern blot, qPCR, in situ FISH. Their statuses are monitored by protein electrophoresis, antibody or histochemistry.

In step (4) of the transgenic cell lines or animals that containing the modified target sequence are given appropriate stimuli that have effect on the status of the introduced target sequences. The gene expression change during these processes is monitored by the phonotypical change. The modified target sequence and its associated factors are crosslinked by crosslink reagents, which include but not limited to formaldehyde, ultraviolet radiation, laser radiation, alkylating agents, reactive chemicals.

In step (5) the crosslinked transgenic cells line or animal tissues sample which contain the modified target sequence are collected. The sample is treated with reagents to make cell lysis, nucleus membrane penetration and cytoplasm content clean. The sample is treated with reagents that can specifically bind and cut the DNA at the introduced cutting sites. Fragments are produced from the modified target sequences. The fragments derived from the modified target sequence are released into appropriate solution, and isolated through centrifuge, ultracentrifuge, and antibody capture or affinity precipitation.

In step (6) the isolated fragments are assayed for the factors associated with it and their binding sites. The fragments are further digested by restriction enzymes to produce smaller fragments. Those small fragments with no binding associated factor are isolated from those small fragments with binding associated factors. The crosslinks in small fragments with associated factors are reversed. The associated factors are assayed by protein assay method, which include but not limited to electrophoresis, western blot or mass spectrum. The small fragments are amplified by PCR and sequenced, their position on the target sequence are located.

### Objects of the Invention

This invention provides a method to screen, isolate and assay the factors that bind to certain locus of the genomic sequence. This method is a technique that assays gene regulation mechanism and relevant factors in vivo at both cellular and whole animal level. This method overcomes the two major challenges in studies of a selected target genomic locus: 1) the difficulty to obtain enough material to assay a selected target genomic locus (each cell usually just has 2 copies of a locus). This is overcome by introducing more than one copy of the target genomic locus into a cell; 2) the difficulty to specifically obtain the selected target genomic locus from the cellular chromatin. This is overcome by selecting and/or introducing unique cutting sites for certain endonuclease or chemical cutter. These selected and/or introduced cutting sites are specifically cut by contacting the endonuclease or chemical cutter, make double strand DNA breaks and produce fragments from the target sequences.

The principle of this invention is based on the following knowledge: current molecular techniques (homologous recombination and gene assembly) are able to introduce defined nucleic acid sequence to any selected target genomic sequence or locus in vitro and in vivo. Those introduced sequence can be recognition sites of certain kind of endonuclease or chemical cutter. By appropriate selection the type of the endonuclease or chemical cutter, the introduced cutting sites will form the unique sites in the whole genome. The modified target sequences are amplified in vitro and purified with standard molecular method. The purified modified target sequences are introduced into cells or animals to produce transgenic cell lines or animals. When the modified target sequence is long enough, it will contain all the regulatory elements and their status are controlled by the endogenous host cellular factors in the same way as the original endogenous host genomic locus. The modified target sequence and the factors associated with it are crosslinked, treated with reagents that specifically bind and cut at the introduced recognition sites and produce fragments from the modified target sequence. The fragments from the modified target sequence with its associated factors are isolated and assayed with standard molecular method to determine the types and amount of the factors and their binding sites.

This method is based on the structure of a eukaryotic cell genome. When a fragment of a genomic locus is large enough, it contains all the elements that it needs to regulate its function. When this fragment is introduced into host cells, its function is regulated by the endogenous host factors in the same way as the host original gene and independent from their genomic position. This method can be extended to assay any locus associated factors in any eukaryotic genome to explore of mechanism of disease and potential therapeutic targets.

### Summary of the Claims

A method to screen, isolate and assay a locus of interest in chromosomal cellular chromatin in cells includes the following steps. In a first step, a user obtains the target sequence of the locus of interest in chromosomal cellular chromatin and/or genomic library clone, which contains the locus of interest. The locus of interest has a length between 10 kb-1 Mb, and the target sequence has a length preferably between 20-200 kb. Then in a second step a user determines the target sequence structure and potential binding sites of associated factors. Then in a third step, a user modifies the target sequence by selecting and/or introducing one or more unique sequences or binding sites that can be used to select the modified target sequence in the following steps. Then in a third step, the user amplifies and purifies the modified target sequence in appropriate cell lines by clonal amplification or through synthesis method. The user then introduces the amplified and purified modified target sequences into appropriate cells or animals. Then in a fourth step, the user monitors the target sequence status. Then in a fifth step, the modified target sequences and the factors associated with them in the transgenic cells are crosslinked by crosslink reagents, the modified target sequences which are bounded by endogenous binding factors are cleaved at the selected and/or introduced specific sites by contacting specific reagents and making double strand DNA breaks and produce fragments from the target sequences. In a final step, the user isolates the crosslinked modified target sequence fragments and the factors associated with them from the transgenic cells or animals, and begin assaying the type, amount, and binding sites of the associated factor in the modified target sequence.

Optionally, in the second step, the sub step of introducing modification in the target sequence includes using a selectable marker and/or reporter marker, which are used to select and monitor the cells that have the introduced modified target sequence. The introducing of the modifications in the target sequence also includes, but is not limited to the introduction of one or more unique cutting sites which can be recognized and bounded by endogenous or exogenous cutting reagents that can cleave and make double strand DNA breaks at the one or more unique cutting sites, wherein the cutting reagents include extreme rare endonuclease, these extreme rare endonucleases include but not limited to meganuclease, recombinase, integrase and TALNs (Transcription Activator-like Effector Nuclease), and chemical cutters. The introduced recognition sites are unique nucleic acid sequences in the whole genome, the introduced recognition sites length is longer than 10 bp, wherein the introduced modifications in the target sequence can be realized with genomic recombination and/or artificial synthesis.

Additionally, the user can amplify the modified target sequences in appropriate cell lines or artificial synthesis, wherein the amplification methods includes, but is not limited to standard clone amplification in clonal vector in appropriate cell lines, and/or Gibson Assembly, or other similar in vitro assembly method. The user can then isolate and purify the amplified modified target sequences using standard molecular methods. The user can then introduce the purified modified target sequences into appropriate cells to prepare transgenic cells or animals. The methods of introducing modified target sequences include, but not limited to cyclodextrin, polymers, liposomes, nanoparticle, calcium phosphate mediated, electroporation, optical transfection, nucleofection and microinjection. The cells or animals which contain the modified target sequences are selected by monitoring the existence of the modified target sequences.

The method optionally further includes the steps of testing the cells or animals which contain the modified target sequences by southern blot, qPCR, in situ FISH and/or protein electrophoresis, antigen specific antibody and histochemistry to detect the status of the modified target sequences. The user can use endogenous and exogenous factors to influence the status of the modified target sequence and the factors associated with it in the host transgenic cells or animals. These factors also influence the modified target sequence endogenous counterpart in the same way. The modified target sequence and the factors associated with them in the host cells or animals are captured by contacting them with crosslink reagents. The crosslink reagents include, but are not limited to: formaldehyde, ultraviolet, aldehydes, psoralens, alkylating agents or other reagents. The appropriate formaldehyde concentration need to be optimized by each target sequence, usually its range is approximately between 0.1-4.0%.

After the transgenic cells or animals tissues which contain the modified target sequence and its associated factors are crosslinked by crosslink reagents; then the user can contact the crosslinked transgenic cells or animal tissues which contain the modified target sequence with cellular break reagents to lysis the cell membrane and penetrate the nucleus membrane. The modified target sequence and the factors associated with it are contact with reagents which specifically bind to the selected and/or introduced cutting sites and cleave the DNA at the selected and/or introduced cutting sites, making double strand DNA breaks. The specifically cutting produces one or more fragments from the modified target sequence and the associated factors on them. Cells release the produced fragments from modified target sequence from the rest of the cell chromatin into solution. The user then isolates and collects the fragments produced from modified target sequences. Isolation and collection methods include, but not limited to: centrifuge, sucrose graduation centrifuge, ultracentrifuge, antibody-magnetic beads and fragment terminal labeling hybridization.

The isolated fragments from the modified target sequence in the host cells are treated with endonuclease which has shorter recognition sites and/or exonuclease. This treatment produces smaller fragments from the isolated modified target sequence fragment. The user can then isolate smaller fragments produced from the isolated target sequence fragment. Those smaller fragments which do not contain binding associated factor are separated from those smaller fragments which contain binding associated factors by standard DNA extraction. The no-binding smaller DNA fragments are amplified and sequenced.

The user can then reverse the crosslink in those smaller fragments which are crosslinked with binding associated factors. The released smaller DNA fragments after reversing treatment are isolated from their associated factors by standard DNA extraction. Methods to assay the associated factors include, but are not limited to: protein electrophoresis, western blot, proteinase digestion, mass spectrum assay, and non-coding RNA assay, wherein the small DNA fragments dissociated from binding associated factors are assayed with DNA extraction, amplification and sequencing. The user then compares the binding factors data and their binding sites sequence data with the target sequence. This alignment assay will give the information about the associated factors type, amount and their binding sites in the target sequence.

### DESCRIPTION OF THE DRAWINGS

FIG. 1 is a flowchart showing the first four steps of the present invention method, with some substeps broken up into separate steps.

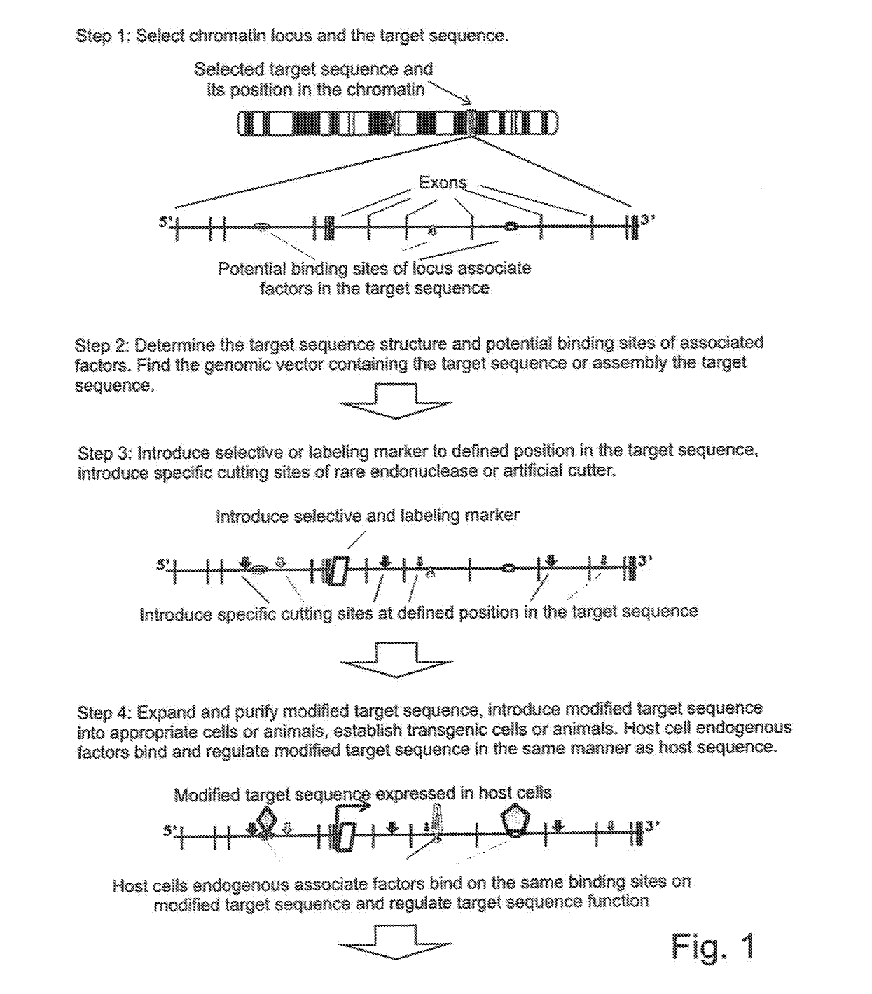

FIG. 2 is a flowchart showing the last four steps of the present invention method, with some substeps broken up into separate steps.

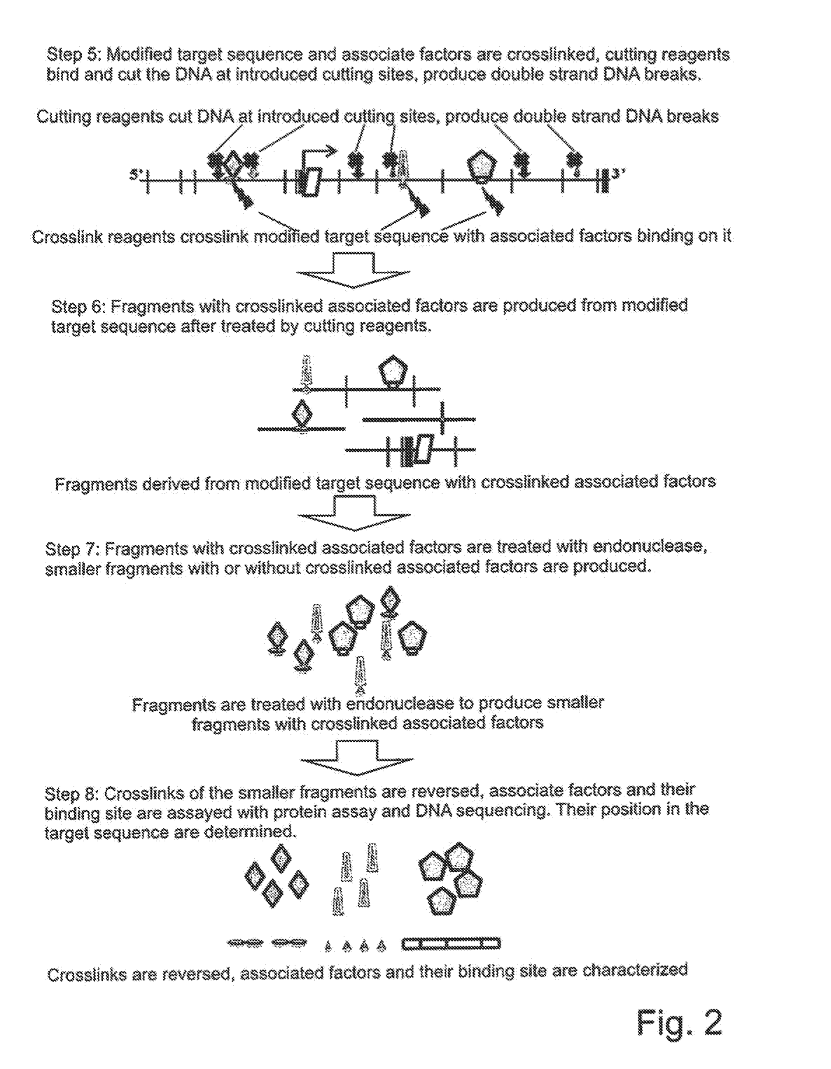

### DETAILED DESCRIPTION OF THE PREFERRED EMBODIMENT

This invention provides a method to screen, isolate and assay the factors associated with a special target gene or locus in eukaryotic genome. The gist of the invention is to increase the locus copy number in cells. Each cell usually has two copies of a locus. Isolating loci typically requires a large amount of original material. The target sequence in the locus is modified, amplified and transfer into cells. These modified target sequences in the transgenic cells will bind to the same sets of associated factors as their endogenous counterpart if they are large enough to contain all the regulatory elements. The introduced modified target sequence will increase the detection sensitivity and decrease the amount of original material required. Using the modified target sequence will also help to isolate it with a high specificity, since unique sequence can be selected and/or introduced into it. This method can be used in studying any locus in the genome such as to study the changes of DNA, RNA and protein.

### Background and Technology Used

Using techniques in molecular biology, the present invention method isolates and assays the factors that associate with a special locus of the chromatin. Endonucleases are enzymes that cleave the phosphodiester bond within a polynucleotide chain. Restriction endonucleases are a small number of significant classes of endonucleases that cleave only at the specific nucleotide sequences and are usually called restriction enzymes. The nucleotide sequence recognized for cleavage by a restriction enzyme is called the recognition site. Typically, a restriction site will be a palindromic sequence of about 4 to 8 nucleotides long. After recognizing and binding with their recognition site in the DNA, restriction enzymes produce a double-stranded the DNA break. When the recognition site of a restriction enzyme becomes longer, their distribution in a genome becomes rare. For example, an 18-base recognition site on average would require a genome twenty times the size of the human genome to be found once by chance.

Meganucleases are endodeoxyribonucleases characterized by a large recognition site (double-stranded DNA sequences of 12 to 40 base pairs), these sites generally occur only once in any given genome. Currently, there are more than 100 type of meganuclease found in different species, and this number keeps growing. Due to recognition sites being very rare in every species' genome, meganuclease become a valuable tool for the high-specific, high-selective tool for gene targeting, including gene therapy, genetic changes. According to their structure analysis, researchers have developed a serial of artificial meganucleases. These artificial meganucleases could almost recognize any specific sequence in genome.

Besides meganucleases, there are other kind of extreme rare restriction enzymes, such as zinc-finger and transcription activator-like effector nucleases (ZFNs and TALENs), site-specific recombinases (SSRs) and integrase, which can make double strand DNA breaks at their recognition site.

Double strand DNA breaks can also be made by artificial chemistry-based DNA cutters which are called artificial cutters. These cutters use a DNA-cutting molecule combined with a sequence-recognizing molecule in a covalent or non-covalent way. At targeted sites, the scission occurs via either oxidative cleavage of nucleotides or hydrolysis of phosphodiester linkages. The specificity are determined simply in terms of the sequence-recognizing molecule, some cutters use the Watson-Crick rule so that even the whole human genome can be selectively cut at one predetermined site.

Since most genes' length in a genome are less than 100 kb, when a fragment of a genome long enough, this fragment will contain all coding sequence of a gene and its regulation non-coding sequences. Genomic vectors with large insert capacities have been developed, many organism genomic library have been constructed from these vectors. For example, 100-300 kb genomic fragments can be inserted into bacterial artificial chromosomes (BAC), the insert size of yeast artificial chromosome (YAC) can reach 500 kb to more than 1 MB. Many genomic library constructions from these genomic vectors have become commercially available. These genomic libraries can supply almost any fragment from the genome.

Many mature molecular methods could be applied to modify the genomic fragments in these genomic vectors, including targeted mutant, insertion, and deletion. These modified genomic fragments can be amplified, isolated and purified readily in vitro in large amount. Commercial genomic engineering services are also available.

Advances in biology synthesis also make it possible to assemble different small DNA fragments together to make a large fragment. The whole assembly process could be done in vitro with high efficiency. With these assembly techniques it becomes feasible to introduce certain sequences to the assembly DNA. The assembly method could produce genomic fragments more than 500 kb, the reagents for these assemblies are also commercially available.

Various techniques are available to introduce large capacity genomic vectors into cells and establish transgenic cells lines and animals. Both BAC and YAC transgenic mice have been reported and large scale projects using these genomic vectors have started. In transgenic cells and animals the large genomic fragments usually contain all the gene coding sequence and its regulation non-coding sequence. The large size of the genomic fragment guarantees that the genes in it are regulated in the same way as their original in vivo partner and independent from the sites they insert into. The gene expression status faithfully reflects the in vivo genes' regulation status and the expression products reflect the copy number of the transgene.

### Steps of the Present Invention

A method is provided for assaying peptide, polypeptide, protein complexes and non-coding factors that are associated with nucleic acid sequences, particularly genomic DNA and chromatin at a defined position.

The first step is to select the chromatin locus and the target sequence. Select the target sequence or genomic locus, through the genomic database or relevant vector library, and then find the vector clones containing the target sequence or locus. The second step is to determine the target sequence structure and potential binding sites of associated factors. Find the genomic vector containing the target sequence or assemble the target sequence. Depending on the purpose of the research, decide the length of the target sequence that will be manipulated (on common principle, this target sequence should include all the potential associated sites, to simplify the following steps, the selected genome sequence length could be 10 kb-1 Mb, usually between 100-300 kb).

Currently, many open resources regarding the genome database am searchable with various data mining tools. For example, the NIH genome website database is available at (http://www.ncbi.nlm.nih.gov/sites/genome). The vector library of large genome fragments can be searched at professional websites and ordered (http://bacpac.chori.org). Other resources for transgenic animal projects can also supply modified genomic fragment clones. Another way is to construct user's own genomic vector library.

The third step is to modify the target sequence. This can include: 1) Select and/or introduce a selectable and/or reporter marker at defined sites in the target sequence. The selectable and/or reporter marker can be antibiotic and/or fluorescence proteins which are introduced to replace partially or entirely encoding sequence of the target sequence. These makers will help to select the positive transgenic cells and animals. 2) Select and/or introduce one or more manipulated sequences at defined sites in the target sequence. The manipulated sequence can be specific cutting sites which include extremely rare endonuclease recognition sites and artificial cutter sites. Extremely rare endonucleases have long recognition sites (more than 10 bp), they include but are not limited to meganuclease, recombinase, integrase and artificially modified enzymes. Meganuclease is a type of endonuclease that includes intron endonuclease and intein endonuclease. Their recognition sites length are around 12-40 bp. Different kinds of endonuclease have different recognition sequences and some of the enzymes are commercially available. Recombinase, integrase and other endonuclease recognition sites length are between 30-200 bp. They can be expressed using gene engineering and have commercial products. Artificially modified enzymes include zinc-finger endonuclease and TALNs. Their recognition size can be designed and longer than 10 bp. Extremely rare endonuclease bind on these introduced sites and cut DNA to produce double strand DNA breaks.

The method of introducing manipulated sequences includes site specific recombinase technology, synthetic biology (gene assembly) and a combination of these two methods. Site-specific recombinase technology use vector carrying recombinase sequence and vector carrying a manipulated sequence flanked with two homologous sequences to the insert site in the target sequence. When these two type vectors are introduced into the same cells and expressed, the homologous recombination starts and the recombinase replace the target site with manipulated sequence.

Synthetic biology, through Gibson assembly method is a DNA assembly method which allows for the joining of multiple DNA fragments in a single, isothermal reaction (See http://www.synbio.org.uk/dna-assembly/guidetogibsonassembly.html). When use a combination two methods, the different target sequence and manipulated sequences fragments could be introduced into different vectors and amplified, purified as standard molecular method, then the fragments can be isolated and assembly through an assembly method.

The fourth step is to amplify and purify the modified target sequence and introduce the modified target sequence into appropriate cells or animals, and establish transgenic cells or animals. Modified target sequences amplification is based on standard molecular techniques. The modified target sequence in appropriate clone vectors are introduced into appropriate host cell lines and amplified according to standard method. Alternatively, the modified target sequence can be produced with gene assembly methods such as Gibson assembly. Standard molecular techniques are used to purify the amplified modified target sequences. Standard transgenic methods are used to introduce the modified target sequence into cell lines or animals.

The methods of introducing modified target sequence into cells include, but are not limited to calcium phosphate, electroporation, or cationic lipid formed liposomes. The host cells containing the modified target sequence are selected by phenotype changes. Transient expression or stable cell lines can be established. The methods of introducing modified target sequence into animals include, but are not limited to DNA microinjection, retrovirus-mediated, and stem cell-mediated techniques.

Modified target sequence function detection in vivo. Host cell endogenous factors bind and regulate modified target sequences as the same manner as the host endogenous target sequence. Standard molecular methods are used to determine the copy number of the modified target sequences in host cells or animals. These methods include, but are not limited to southern blot, quantitative PCR, in situ FISH. The host cells or animals containing the modified target sequence are monitored for phenotype changes after receiving appropriate stimuli. These stimuli include, but are not limited to medicine, physical stimulus, chemical stimulus, and biological stimuli. The status of the modified target sequence in the host cells or animals are monitored using standard molecular, biochemistry, and histology methods. These methods include, but are not limited to protein electrophoresis, antigen specific antibody detection, or histochemistry.

The fifth step is to cross-link the modified target sequence and associated factors. The host cells or animal tissue sample that contain the modified target sequence are collected, crosslink reagents are added into the sample, the modified target sequence and the factors associated with it are crosslinked together. The crosslink reagents include, but are not limited to formaldehyde, ultraviolet radiation, laser radiation, alkylating agents, and reactive chemicals.

The sixth step is to produce fragments with cross-linked associated factors from modified target sequence after treatment by cutting reagents. The crosslinked host cells or animal tissues sample that contain the modified target sequence are treated with standard molecular method to break the cellular membrane, nucleus membrane and cytoplasmic content. These methods include, but are not limited to detergents treatment, cell lysis, and nucleus penetration. The nucleus of the crosslinked host cells or animals tissue sample which contains the modified sequence are treated with one or more cutting reagents, which can specifically bind and cut at the introduced cutting sites in the modified target sequence. The cuttings make double strand DNA breaks in the modified target sequence and produce one or more small fragments from the modified target sequence. The cutting reagents include, but are not limited to megaendonuclease, integrase, recombinase, zinc-finger endonuclease, TAL nucleases, or chemical cutter.

The seventh step seven is to treat the fragments with cross-linked associated factors with one or more endonuclease which have shorter recognition site (4-6 bp). This treatment produces smaller fragments with or without cross-linked associated factors.

The eighth step is to reverse the cross-links of the smaller fragments obtained in step seventh. This treatment release the associated factors from their binding fragments. The binding associated factors are separated assayed with standard protein assay, which include but not limited to: protein electrophoresis, westernblot, peptide assay, and mass spectrum. The shorter fragments freed of associated factors are amplified with PCR and sequenced. The shorter fragments positions in the target sequence are determined.

### EXAMPLE

Unless otherwise defined, all technical and scientific terms used herein have the same meaning as commonly understood by one of ordinary skill in the art to which this invention belongs. The present invention involves use of a range of conventional molecular biology techniques, which can be found in standard texts such as Sambrook et al. (Sambrook et al (2001) Molecular Cloning: A Laboratory Manual; CSHL Press USA). Most steps in this example are followed the protocol provided by references:
Nat Methods, 2008; 5(5);409-15. BAC TransgeneOmies: high-throughput method or exploration of protein function in mammals.
BAC modification also provided by commercial service supplier.

### Step 1: Preparation of a Target Sequence

A 40 kb human genomic fragment located at the upstream of gene actin is obtained from a self BAC construction library. A BAC clone with this insert of 40 kb of human DNA has G418 resistance. Two I-Ceul homing endonuclease recognition site (5′TAACTATAACGGTCCTAA GGTAGCGA 3′ATTGATATTGCCAG GATTCCATCGCT) are introduced into the middle of the 40 kb genomic fragment with one step homologous recombination using with 2 sets of 50 bp homologous arms flanking the I-Ceul sequence. The target sequence between the two I-Ceul recognition sites is 5 kb. The positive clone is picked up with PCR verification with 2 sets of primers cover the 2 insertion sites.

Positive clones are expanded according to standard method. Inoculate a fresh colony of modified BAC into 50 ml of LB media supplemented with chloramphenicol and kanamycin and grown overnight at 37° C. Harvest bacteria from LB culture (OD600˜1.8-2.0) by centrifugation at 4,500 g for 15 min at 4° C. and discard the supernatant. Proceed with BAC isolation by following the protocol “Low-copy plasmid purification: Maxi/BAC” of the “Nucleobond AX 100 kit, 10 μl of the isolated BAC on a 0.8% agarose gel (70V, 1 h) to verify good quality of the BAC isolation. For best transfection results, isolated BACs need to be of high quality. A large fraction of supercoiled BAC is especially important.

Step 2: Transfect BAC into Mammalian Cells

HeLa cells are cultured in DMEM/Glutamax (4.5 g glucose/500 ml, Invitrogen) supplemented with 10% FCS (Hyclone), 100 units/ml Penicillin, and 100 μg/ml Streptomycin (Gibco). Lipofectamine 200 are used as transfection reagents. Plate 200,000 cells into tissue-culture dishes (60 mm). Use one dish for modified BAC transfection. Two more plates are to transfect an unmodified BAC (negative control) and a verified BAC serves as a positive transfection control. Prepare the transfection mix for each BAC to be transfected using a separate 1.5 ml cup. Transfection is performed according to the manufacturer's protocol supplied with each transfection reagent. Add the entire transfection mix drop-wise to the cells and mix by gently rotating the whole dish horizontally. Change the complete cell culture media the next day. Two days after transfection, change media and culture the cells in complete media supplemented with G418. After 2 weeks, distinct stable colonies are visible in the cell dishes transfected with modified BACs.

Step 3: Crosslink the Cells

Stable HeLa colony cells (1X10 8) are produced. The media was first discarded and crosslinking solution is immediately added to the plates (10 ml/15 cm plate). Cells were incubated in crosslinking solution for 30 minutes at room temperature. The crosslinking solution was discarded and the plates washed twice with 1×PBS solution (standard phosphate buffered saline solution supplemented with 1 mM PMSF). A further 3 ml of cell scrapping solution (1×PBS; 0.05% Tween-20) was added per plate and the cells were pooled into Falcon tubes on ice. The cells are then washed four times in PBS by resuspending the cell pellet in 1×PBS solution bringing the volume to 50 ml/tube, then spinning down at 3200 g for 10 minutes at 4° C. The supernatant is discarded each time and the final washed pellet was resuspended in sucrose solution (bringing the volume to 50 ml/tube). The solution is spun down at 3200 g for 10 minutes at 4° C., the supernatant is discarded and the pellet brought up to a volume of 20 ml with PBS.

Step 4: Release the Modified Target Sequence from Crosslinked Cells

Cells are washed three times with 1×PBS, then washed 2 times with digestion buffer (50 mM) Potassium Acetate, 20 mM Tris-acetate, 10 mM Magnesium Acetate, 100 μg/ml BSA). Cells are suspended in digestion buffer with a 1:4 volume ratio. Digestion is initiated by adding I-CueI (New England Biolab) to the digestion buffer at the final concentration according to the manufacturer's instruction and incubated for 4 hours at 37° C. with occasional shaking. After spinning down at 3200 g for 10 minutes at 4° C., the supernatant is collected. Another round of digestion carried out as the same way and the supernatant is collected and pooled. The cells are kept on ice with adding PBS.

Step 5: Assay the Fragment from Target Sequence

The collected supernatants are concentrated with Chemicon Protein-Concentrate kit (cat 2100), and the recovered sample is reversed by incubation for two hours at 65° C. in crosslink reversal buffer (10 mM NaOAc pH 5.5; 30 mM NaCl; 0.5 mM EDTA pH 8; 0.1 mM EGTA pH 8; 10 mM Hydrazine (from 11 M stock, neutralized with AcOH); 1% SDS) and in a thermomixer shaking at 1200 rpm.

The proteins are concentrated one more time with Chemicon Protein-Concentrate kit and loaded on a Bis-Tris 12% acrylamide minigel (Invitrogen) and run at 100V until the loading dye exited the gel. The gel is then fixed stained with Colloidal Blue (Invitrogen) following manufacturer's instructions, 15-25 bands are cut all along the lane (covering the whole lane). These samples are submitted to mass spectrometry for analysis and protein identification. Typically this analysis involves the following steps: (a) In-gel digestion of gel bands/spots; (b) Micro-capillary LC/MS/MS anaylsis; and (c) Protein database searching. The peptides identified from the sample, along with the corresponding proteins they matched to, are scored. Proteins that had only one matching peptide are listed for further analysis. Proteins that only had one matching peptide may be correct but are typically verified by further confirmation, such as by western-blot for instance.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes. Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. All the variation designs and/or experiments based on the basic principle of this method, all the factors obtaining through this method and the further development derivatives based on this method findings includes, include but are not limited to small molecules, peptide, polypeptide and RNA.

1. A method to screen, isolate and assay a region of interest in chromosomal cellular chromatin cells comprising the following steps:
a in a first step, obtaining the target sequence of the region of interest in chromosomal cellular chromatin and/or genomic library clone, which contains the region of interest, wherein the region of interest has a length between 10 kb-1 Mb, wherein the region of interest has a length between 20-200 kb; then
b. in a second step determining the target sequence structure and potential binding sites of associated factors; then
c. in a third step, modifying the target sequence by selecting and/or introducing one or more unique sequences or binding sites that can be used to select the target sequence, monitor the target sequence status, bind endogenous or exogenous binding factors which can cleave the target sequence at the introduced specific binding sites and make double strand DNA breaks; then
d. in a fourth step, amplifying and purifying the modified target sequence in part in appropriate cell lines by clonal amplification or through synthesis method, then introducing the amplified and purified modified target sequence in part in appropriate cells or animals; then
e. in a final step assaying the crosslinked modified target sequence and the factors associated with it, isolated from the cells or animals hosts, and assaying the type, mount, and binding sites of the associated factors in the modified target sequence.

2. The method of claim 1, wherein further including the sub step of:
a. in the second step, the sub step of introducing modification in the target sequence includes using a selectable marker and/or reporter marker, which are used to select and monitor the cells that have the introduced modified target sequence, wherein the introducing of the modifications in the target sequence also includes, but is not limited to the introduction of one or more unique cutting sites which can be recognized and bounded by endogenous or exogenous cutting reagents that can cleave and make double strand DNA break at the one or more unique cutting sites, wherein the cutting reagents include to extreme rare endonuclease, which includes meganuclease, recombinase, integrase and TALNs (Transcription Activator-like Effector Nuclease), and chemical cutters, wherein the introduced recognized sites are unique nucleic acid sequences, whose length is longer than 10 bp, wherein the introduced modifications in the target sequence can be realized with genomic recombination and/or artificial synthesis.

3. The method of claim 2, further comprising the steps of:
a. amplifying the modified target sequences in appropriate cell lines or artificial synthesis, wherein the amplification methods includes, but is not limited to standard clone amplification in clonal vector in appropriate cell lines, and/or Gibson Assembly, or other similar in vitro assembly method; then
b. isolating and purifying the amplified modified target sequences using standard molecular methods; then
c. introducing the purified modified target sequences into appropriate cells to prepare transgenic cells or animals, wherein the methods of introducing modified target sequence include but are not limited to: cyclodextrin, polymers, liposomes, nanoparticle, calcium phosphate, electroporation, optical transfection, nucleofection and microinjection, wherein the cells or animals which contain the modified target sequences are selected by monitoring the existence of the modified target sequences.

4. The method of claim 3, further comprising the steps of:
a. testing the cells or animals which contain the modified target sequences by southern blot, qPCR, in situ FISH and/or protein electrophoresis, antigen specific antibody and histochemistry to detect the status of the modified target sequences;
b. using endogenous and exogenous factors to influence the status of the modified target sequence and the factors associated with it in the host cells or animals, in the same way as their endogenous counterpart; and capturing the modified target sequence and the factors associated with them in the host cells or animals by contacting them with crosslink reagent; wherein the crosslink reagents include, but are not limited to formaldehyde, ultraviolet, aldehydes, psoralens, alkylating agents or other reagents, wherein the formaldehyde concentration has a range of approximately 0.1-4.0%.

5. The method of claim 4, further comprising the steps of:
a. after the transgenic cells or animals tissues which contain the modified target sequence and its associated factors are crosslinked by crosslink reagents; then
b. contacting the crosslinked transgenic cells or animals tissues which contain the modified target sequence with cellular break reagents to lysis the cell membrane and penetrate the nucleus membrane and clean cytoplasmic content, wherein the modified target sequence and the factors associated with it are contact with factors which selectively bind to the introduced cutting sites and cleave the DNA at the introduced cutting sites, making the double strand DNA breaks, wherein the cutting produces one or more fragments from the modified target sequence and the associated factors on them; whereby modified host cell chromatins are not kept intact;
c. releasing the produced filaments from modified target sequence from the rest of the cell chromatin into solution;
d. isolating and collecting the produced fragments from modified target sequences in solution, wherein an isolation and collection method includes, but is not limited to centrifuge, sucrose graduation centrifuge, ultracentrifuge, antibody-magnetic beads and fragment terminal labeling hybridization isolation.

6. The method of claim 5, further comprising the steps of:
a. producing an isolated fragment from the modified target sequence in the host cells treated with endonuclease which has shorter recognition sites and/or exonuclease, and producing smaller fragments from the isolated modified target sequence fragment;
b. isolating smaller fragments produced from the isolated target sequence, which do not contain binding associated factor, from fragments which contain binding associated factors by standard DNA extraction, wherein the smaller fragments are amplified and sequenced;
c. reversing the crosslink reagents in the smaller fragments which crosslink binding associated factors, wherein the released smaller fragments are isolated from their associated factors by standard DNA extraction, wherein methods to assay the associated factors include, but are not limited to: protein electrophoresis, western blot, proteinase digestion, mass spectrum assay, and non-coding RNA assay, wherein the small fragments dissociated from binding associated factors are assayed with DNA extraction, amplification and sequencing;
d. taking data and then comparing the data with target sequence, so that the associated factors type, amount and their binding sites are determined in the target sequence, wherein the binding sites location and sequence are determined in the target sequence.
